(19) United States
(12) Patent Application Publication
(10) Pub. No.: US 2022/0331109 A1
(43) Pub. Date: Oct. 20, 2022

(54) COMPOSITE FILAMENT FOR 3D PRINTING OF RESORBABLE BONE SCAFFOLDS

(71) Applicant: Arizona Board of Regents on Behalf of the University of Arizona, Tucson, AZ (US)

(72) Inventors: John A. Szivek, Tucson, AZ (US); Luis Fernando Arciniaga, Tucson, AZ (US); Douglas Loy, Tucson, AZ (US); David Margolis, Tucson, AZ (US); Anupama Peiris, Tucson, AZ (US)

(21) Appl. No.: 17/734,826

(22) Filed: May 2, 2022

Related U.S. Application Data
(63) Continuation-in-part of application No. PCT/US2020/058586, filed on Nov. 2, 2020.
(60) Provisional application No. 62/929,419, filed on Nov. 1, 2019.

(51) Int. Cl. A61F 2/28; B29C 64/118; B33Y 10/00; B33Y 70/10; B33Y 80/00

(52) U.S. Cl. CPC A61F 2/28; B29C 64/118; B33Y 10/00; B33Y 70/10; B33Y 80/00; B29K 2067/046

(57) ABSTRACT

Disclosed are composite filaments for 3D printing. The filaments typically have high strength, an appropriate resorption rate, and high biocompatibility. The filaments generally contain a matrix formed of a blend containing a bioresorbable polymer and an inorganic component. The filaments can be used to produce customized scaffolds for repairing bone defects following implantation in the site of the defect. The shape and size of the scaffold can be configured to fit in and conform to the bone defect. The scaffolds are especially useful in repairing critical sized bone defect, such as a critical sized bone defect in a weight-bearing long bone.

### CROSS-REFERENCE TO RELATED APPLICATION

This application claims the benefit of and priority to U.S. Ser. No. 62/929,419 filed Nov. 1, 2019, which is incorporated by reference herein in its entirety.

### FIELD OF THE INVENTION

The disclosed invention is generally in the field of 3D printing, and specifically in the area of 3D printing of artificial scaffolds for bone repair.

### BACKGROUND OF THE INVENTION

Long bone segment defects in patients typically result from extensive bone resection due to cancer or severe extremity trauma. When these defects are in a weight-bearing long bone, the bone often fails to heal properly following surgical reconstruction, resulting in a nonunion (Keating et al., J Bone Joint Surg Br, 2005, 87(2):142-150). This type of defect is challenging to treat and can lead to amputation or endoprosthetic replacement. Patients with these defects often underwent multiple surgeries, with outcomes of reconstruction with graft compared to amputation being similar, although neither of the results were good.

All of the existing techniques for treating these defects are associated with long-term problems. See, e.g., Chmell et al., Structural allografts for reconstruction of lower-extremity open fractures with 10 centimeters or more of acute segmental defects, J Orthop Trauma, 1995, 9(3):222-226. One common current approach involves the use of long allograft segments held in place with either a rod or plates, and screws, but this approach can fail because healing and remodeling of the allograft do not progress quickly enough. Another common current technique includes vascularized bone grafts of the patient's own tissue. See, e.g., Nusbickel, Vascularized autografts for reconstruction of skeletal defects following lower-extremity trauma: A review, Clin Orthop Relat Res, 1989, 243:65-70. However, placement of these autografts is technically challenging with donor site morbidity. Further, bone graft size mismatching can lead to fracture and failure of this surgical treatment. Keating et al. (2005). Allografts do not require the surgeon to create a second surgical site but have a high risk of long-term postoperative fracture due to incomplete healing, risk of postoperative infection from the donor tissue, and do not contain osteoinductive factors or stem cells. See Delloye et al., Bone allografts: What they can offer and what they cannot, J Bone Joint Surg Br, 2007, 89(5):574-579; Salgado et al., Bone tissue engineering: State of the art and future trends, Macromol Biosci, 2004, 4(8):743-765.

An alternative method, the induced membrane technique, uses a temporary cement spacer, which is implanted for several weeks and replaced with bone graft during a subsequent surgical procedure. This has numerous drawbacks including a prolonged limited weight bearing, a second surgery, donor site morbidity incurred with the bone graft harvest, and the risk that the bone graft will not incorporate. Yet another option is the use of bone transport distraction osteogenesis, which involves an extended use of external fixation, often causing pin site infections, adjacent joint stiffness, and issues with contracture formation following long-segment reconstruction.

Another alternative approach involves regenerating bone in situ to fill these large defects utilizing customizable scaffolds that induce rapid bone formation. However, previous studies using this approach had limited success due to the long time period required for bridging the defect gap. The customizable scaffolds were generally unable to completely bridge critical sized defects within a clinically appropriate time period. See Bostman et al., Foreign-body reactions to fracture fixation implants of biodegradable synthetic polymers, J Bone Joint Surg Br, 1990, 72(4):592-596; Oest et al., Quantitative assessment of scaffold and growth factor-mediated repair of critically sized bone defects, J Orthop Res, 2007, 25(7):941-950.

There is a need for improved treatment methods directed to rapid bone repair and regeneration, especially for critical sized bone defects. There is also a need for improved bone repair materials, particularly for use in repair and regeneration of bone in critical sized bone defects.

It is an object of the invention to provide improved bone repair materials, for forming customizable bone repair scaffolds.

It is another object of the invention to provide improved customizable bone scaffolds for bone repair and/or regeneration.

It is yet another object of the invention to provide methods of making customizable bone scaffolds.

It is yet another object of the invention to provide improved methods for treating bone defects, especially critical sized bone defects.

### BRIEF SUMMARY OF THE INVENTION

Composite filaments for 3D printing bioresorbable scaffolds are disclosed herein. The filaments typically have high strength, an appropriate resorption rate, and high biocompatibility. The filaments can be used in a 3D printer to produce customized bone scaffolds for implantation in a site in need of treatment. The resulting scaffolds typically have high strength, a customizable resorption rate, and excellent biocompatibility.

The composite filaments generally contain a matrix formed of a blend containing a bioresorbable polymer and an inorganic component. Preferably, the blend contains greater than 5% of the inorganic component by weight of the total mass of the matrix.

The bioresorbable polymer in the composite filament can be a polyaklene, polyester, polyurethane, polyurea, polyanhydride, polyamide, nylon 2 or higher nylon (such as nylon 6, nylon 12, and nylon-6,6), or a blend or a copolymer thereof. In some embodiments, the polymer is poly(lactic acid) (PLA). In some embodiments, the polymer is thermoplastic polyurethane. In some embodiments, the polymer is a blend of PLA and thermoplastic polyurethane.

The inorganic component in the composite filaments can be a ceramic, such as a calcium phosphate ceramic. Preferably, the inorganic component contains a tricalcium phosphate, a hydroxyapatite such as a synthetic hydroxyapatite, or a mixture thereof. In some embodiments, the inorganic component contains beta tricalcium phosphate.

Exemplary composite filaments contain PLA, thermoplastic polyurethane, or a blend thereof as the bioresorbable polymer, and beta tricalcium phosphate as the inorganic component.

The composite filament can be used to produce customized scaffolds via 3D printing. The scaffolds can be used for repairing bone defects following implantation in the site of the defect. The shape and size of the scaffold can be configured to fit in and conform to the bone defect. Preferably, the scaffolds are porous, allowing cells to grow throughout the scaffolds for effective, rapid bone regeneration.

Methods of treating a bone defect in a patient are also disclosed. The methods generally include implanting a scaffold in the patient to repair a bone defect. The methods optionally include seeding the scaffold with stem cells derived from the patient, prior to implantation. The bone defect may be a critical sized bone defect. The bone defect may be in a weight-bearing bone.

### BRIEF DESCRIPTION OF THE DRAWINGS

FIG. 1 shows images of exemplary biomimetic, 3D-printed scaffolds made from PBT (left), PLA (middle), and 50:50 PLA/TCP (right), illustrating that they have the same size and structure.

FIG. 2 is a bar graph showing the compressive modulus (in MPa) of the scaffold materials. Empty bars: before cell culture soaking; filled bars: after 4-week cell culture soaking.

FIG. 3 is a bar graph showing the number of viable cells in the cell culture media. Black bars: cell counts at 7 days; gray bars: cell counts at 14 days. The error bars were calculated based on results from six parallel scaffolds in culture.

FIG. 4 is a bar graph showing the alkaline phosphatase (ALP) activity (in units/ml/cell) in the cell culture media. Black bars: ALP activity at 7 days; gray bars: ALP activity at 14 days. The error bars were calculated based on results from six parallel scaffolds in culture.

### DETAILED DESCRIPTION OF THE INVENTION

Composite filaments can be created by combining two or more materials to form a filament. The composite filament can include more than one type of polymer or a blend of more than one material group, e.g., a polymer-ceramic composite filament. The materials in the filament are selected to provide the desired mechanical and chemical properties.

The composite filaments can be 3D-printed to form customized scaffolds with the desired size and shape and properties, such as bone ingrowth and scaffold resorption rates, to promote bone growth in different musculoskeletal sites.

The composite filaments can contain a polymer that is rapidly resorbable in the body mixed with an inorganic component that is slowly resorbable. Preferably, the composite filaments can attract or activate specific cells (e.g., stem cells, progenitor cells, osteoblasts, osteocytes) causing them to begin rapidly forming tissue.

### I. Definitions

A critical sized bone defect is not expected to heal spontaneously during a patient's lifetime. For example, bone loss greater than 2 times the diameter of the long bone diaphysis is unlikely to result in union despite currently considered appropriate stabilization methods.

As used herein, the term “biocompatible” refers to materials that are, along with any metabolites or degradation products thereof, generally non-toxic to the recipient, and do not cause any significant adverse effects to the recipient, at concentrations resulting from the degradation of the administered materials. Generally speaking, biocompatible materials are materials that do not elicit an inappropriate inflammatory or immune response when administered to a patient.

“Bioresorbable” generally refers to a material or a scaffold that can be absorbed by the body so that the components from which it is made dissolve, degrade, and/or are absorbed by the body.

The terms “treatment” and “treating” refer to the medical management of a subject with the intent to cure, ameliorate, stabilize, or prevent one or more symptoms of a disease, pathological condition, or disorder. This term includes active treatment toward the improvement of the disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization, or prevention.

Numerical ranges can include ranges weight percentages, ranges of integers, ranges of temperatures, ranges of times, ranges of diameters, etc. The disclosed ranges of any type, disclose individually each possible number that such a range could reasonably encompass, as well as any sub-ranges and combinations of sub-ranges encompassed therein. For example, disclosure of a thickness range, is intended to disclose individually every possible thickness value that such a range could encompass, consistent with the disclosure herein. In another example, the disclosure that a temperature is in the range of about 150° C. and about 270° C. also discloses that the temperature can be selected independently from about 155, 180, 200, 225, 260° C., or any individual temperature value within the range, as well as any sub-range between these numbers (for example, about 160° C. to about 240° C.), and any possible combination of ranges between these temperature values.

The term “about” or “approximately” as used herein generally means within 20%, preferably within 10%, and more preferably within 5% of a given value or range. The term “about” modifies all values and ranges immediately following the term, unless stated otherwise.

### II. Composite Filament

The composite filaments disclosed herein can be used in a 3D printer to form a bioresorbable scaffold suitable for implantation in a patient. The composite filaments generally contain a matrix formed of a mixture or blend containing a bioresorbable polymer and an inorganic component. Preferably, the concentration of inorganic component in the composite filament is greater than 5% by weight of the total mass of the matrix. Optionally, the composite filaments do not contain collagen.

The bioresorbable polymer and the inorganic component can be mixed in a range of ratios, which affect both the stiffness and resorption rate of the composite filaments and the customizable scaffolds made from the composite filaments. This allows tailoring of the composite filaments to form scaffolds that are suitable for particular bone defect locations. For example, in a region that requires long-term structural support like the tibia, the ratio of the bioresorbable polymer to the inorganic can be selected such that the resulting scaffolds are resorbed slowly and retain strength for a long period of time. In a region that requires short-term strength, such as a hand bone or finger, the ratio of the bioresorbable polymer to the inorganic can be selected such that the resulting scaffolds are resorbed quickly even though it will lose strength in a short period of time. The weight ratio of the bioresorbable polymer to the inorganic component in the composite filaments can be in the range between about 20:1 and about 1:1, between about 10:1 and about 1:1, between about 5:1 and about 1:1, between about 2:1 and about 1:1, or about 1:1.

Optionally, the composite filaments include a coating. In some embodiments, the coating is formed of tricalcium phosphate particles, optionally the particles have a diameter of about 5 microns. In some other embodiments, the composite filaments are not coated with a ceramic (e.g., tricalcium phosphate).

Preferably, the composite filaments are elastic in their mechanical response.

The composite filaments may have a melting point between about 150° C. and about 270° C. The composite filaments may have a diameter between about 1.5 and about 2 mm, preferably between about 1.65 mm and about 1.8 mm optionally the diameter is 1.75 mm or about 1.75 mm. The diameter of the composite filaments is suitable to allow for printing on commercially available 3-D printers. In some instances, the composite filament can have a diameter that is smaller than about 1.75 mm, such as 1.65 mm, but for use in commercial 3-D printers, the user may have to manually push the composite filament through the 3-D printer if the motor of the printer does not catch the filament.

The composite filaments may have any suitable length that allows for printing on commercially available 3-D printers. In some instances, the filaments can be made in straight lengths of up to about one meter so they can be fed into a 3-D printer, as needed. In some instances, the composite filaments are wound onto a spool; typically a uniform heating element is used to keep the composite filament from solidifying while it is being wound onto a spool. The filament on the spool can then be used to feed the filament into a 3-D printer to print the scaffolds described herein.

A. Bioresorbable Polymer

The composite filaments can contain one or more bioresorbable polymers. The bioresorbable polymer allows the resulting scaffold to degrade and/or be absorbed following implantation. Preferably, the bioresorbable polymer has a low water solubility so that the scaffolds are not rapidly disintegrated after implantation. In some embodiments, the solubility of the bioresorbable polymer is in the very slightly soluble range (1,000 to 10,000 mass part of water required to dissolve 1 mass part of the polymer) or the practically insoluble or insoluble range (>10,000 mass part of water required to dissolve 1 mass part of the polymer), according to the Pharmacopeia of the United States of America, 32nd revision, and the National Formulary, 27th edition. Preferably, the bioresorbable polymer is elastic in its mechanical response.

The bioresorbable polymer is biocompatible.

In some embodiments, the bioresorbable polymer is biodegradable. For example, the polymer can degrade or erode by enzymatic action or hydrolysis under physiologic conditions to smaller units or chemical species that are capable of being metabolized, eliminated, or excreted by a subject.

Suitable polymers can include, but are not limited to, poly(lactic acid), thermoplastic polyurethane, polyalkenes, polyureas, nylons, poly(hydroxy acids), polyurethanes, polyanhydrides, polyorthoesters, polyesters, poly(ester amides), polyamides, poly(ester ethers), polycarbonates, polyvinyl ethers, polyvinyl esters, polyvinyl halides, polysiloxanes, polyhydroxyalkanoates, poloxamers, polyphosphazenes, polymers formed from lactones, celluloses, alkyl celluloses, hydroxyalkyl celluloses, cellulose ethers, cellulose esters, nitro celluloses, hydroxypropylcellulose, carboxymethylcellulose, poly(glycolic acid), poly(lactide-co-glycolide), polydioxanone, polypropylene fumarate, poly(butic acid), poly(valeric acid), polycaprolactone, ethylene vinyl acetate polymer, polyalkyl cyanoacrylate, poly(hydroxybutyrate), poly(lactide-co-caprolactone), poly(ethylene-co-maleic anhydride), poly(ethylene maleic anhydride-co-L-dopamine), poly(ethylene maleic anhydride-co-phenylalanine), poly(ethylene maleic anhydride-co-tyrosine), poly(butadiene-co-maleic anhydride), poly(butadiene maleic anhydride-co-L-dopamine), poly(butadiene maleic anhydride-co-phenylalanine), poly(butadiene maleic anhydride-co-tyrosine), poly(fumaric-co-sebacic anhydride), and blends and copolymers thereof.

Optionally, the bioresorbable polymer is a polyaklene, polyester, polyurethane, polyurea, polyanhydride, polyamide, nylon 2 or higher nylon (such as nylon 6, nylon 12, and nylon-6,6), or a blend or copolymer thereof. In some embodiments, the bioresorbable polymer is PLA. In some embodiments, the bioresorbable polymer is thermoplastic polyurethane. In some embodiments, the bioresorbable polymer is a blend of PLA and thermoplastic polyurethane.

The amount of any one bioresorbable polymer in the filament may be between about 5 wt % and about 95 wt %, between about 15 wt % and about 85 wt %, between about 25 wt % and about 75 wt %, between about 35 wt % and about 65 wt %, or between about 45 wt % and about 55 wt %, by weight of the matrix of the composite filaments.

Optionally, the bioresorbable polymer can be part of a block copolymer, which also contain one or more blocks that are not bioresorbable. Optionally, the bioresorbable polymer is a block copolymer that includes a bioresorbable polymer, and optionally contains one or more blocks that are not bioresorbable.

Optionally, the bioresorbable polymer is elastic in its mechanical response.

B. Inorganic Component

The composite filaments contain a biocompatible inorganic component, which mimics the inorganic component (e.g., minerals) of the bone. The inorganic component can adjust the biophysical properties of the composite filaments as well as the customizable scaffolds made from the composite filaments. For example, the inorganic component can increase the strength of the filaments and resulting scaffolds, and/or increase their stress-bearing capabilities compared to the same filaments or scaffolds in the absence of the inorganic component. Further, the presence of the inorganic component in the resulting scaffold provides a scaffold with properties similar to the bone surrounding the site of implantation. Thus, the scaffolds can enable cell attachment and growth, and thus promote rapid osteogenesis after implantation of the customizable scaffolds.

Optionally the inorganic component is in the form of particles. Particles can have any suitable shape, such as spherical. In some instances, the particles have sizes within a range of about 1 to about 25 microns or about 5 to about 20 microns. Populations of the particles may be monodisperse or polydisperse.

The inorganic component can be a ceramic. The ceramic can be an inorganic, non-metallic solid material made of a metal or a non-metal compound. Optionally the ceramic is crystalline or non-crystalline. Optionally, the ceramic is an oxide. For example the inorganic component can be calcium phosphate ceramic. The calcium phosphate ceramic may contain one or more of the following calcium phosphates: monocalcium phosphate, dicalcium phosphate, tricalcium phosphate, octacalcium phosphate, amorphous calcium phosphate, or dicalcium diphosphate (e.g. dicalcium phosphate anhydrous, dicalcium phosphate dihydrate), calcium triphosphate, hydroxyapatite, apatite, carbonated apatite, calcium pyrophosphate, a hydroxyapatite/calcium carbonate mixture, biphasic calcium phosphate, β-tricalcium phosphate, and tetracalcium phosphate.

Preferably, the inorganic component contains a tricalcium phosphate, a hydroxyapatite such as a synthetic hydroxyapatite, or a mixture thereof. In some embodiments, the inorganic component contains beta tricalcium phosphate.

The inorganic component may contain other materials, such as silica, alumina, calcium carbonate, and calcium sulfate. The amount of the other materials may be up to about 10% by volume or up to 20% by volume of the total volume of the matrix.

Preferably, the amount of the inorganic component may be greater than 5%, greater than 10%, or greater than 20% by weight of the total mass of the matrix. Exemplary amounts of the inorganic component can be about 15%, about 25%, or about 50% by weight of the total mass of the matrix.

### III. Scaffolds

The composite filament can be used to produce customized scaffolds via 3D printing. The scaffolds are biocompatible. The scaffolds can be implanted in a patient at the site of a bone defect to induce bone growth at the site, and optionally repair the bone defect.

The scaffolds may have a compressive modulus between about 40 MPa and about 200 MPa, between about 40 MPa and about 150 MPa, between about 40 MPa and about 150 MPa, between about 50 MPa and about 100 MPa, or between about 50 MPa and about 90 MPa. The scaffolds may have a maximum compression or tensile load of at least 1,500 N, at least 2,000 N, or at least 2,500 N.

The shape and size of the scaffolds can be configured to fit in and conform to the bone defect. For example, the shape and size of the scaffolds can be determined based on the configuration and pattern of the bone defect, which can be visualized using imaging techniques such as radiography, CT scanning or μCT scanning.

The scaffolds are bioresorbable. Depending on the composition of the composite filaments, the scaffolds can have a range of resorption rates. For example, the scaffolds can be completely resorbed within two weeks, within one month, within two months, within three months, or within six months after implantation. In some embodiments, the scaffolds are not completely resorbed even after a longer period of time following implantation. In some embodiments, one or more portions of the scaffolds will remain unresorbed six months, nine months, one year or later following implantation.

Optionally, the scaffolds are porous, allowing cells to grow throughout the scaffolds for effective bone regeneration. In some instances, the scaffold has uniform porous channels, while in others the porous scaffold contains non-uniform porous channels. The porous channels are created in the 3-D printing process for forming the scaffolds and can be tuned/varied as desired. The uniform porous channels in the printed scaffolds have diameters as small as approximately 400 μm, in some embodiments the uniform porous channels have diameters greater than 500 μm. However greater diameters are possible, such as diameters of 550 μm or greater, 600 μm or greater, 650 μm or greater, 700 μm or greater, 750 μm or greater, 800 μm or greater, etc. In still other instances, porous can refer to the presence of uniform or non-uniform pores within the scaffolds which are microporous having a suitable pore size (if uniform in size) or range of pore sizes (if non-uniform in size) in the range of for example, about 100 to 500 micrometers.

Exemplary scaffolds can be 3D-printed from composite filaments that contain PLA, thermoplastic polyurethane, or a blend thereof as the bioresorbable polymer, and beta tricalcium phosphate as the inorganic component.

The scaffolds (such as PBT, PLA, or composite ceramic polymer biomimetic scaffolds) can be mechanically tested/evaluated to determine and compare their mechanical properties. For example, the scaffolds can be affixed to a control bone in the same location and orientation using any suitable material, such as an adhesive or glue, such as an epoxy. The bones can be potted in a test fixture using, for example, Cerrobend so that a strain gauge can be aligned with the loading axis of the test fixture. The bones can then be loaded at least six times at a rate of, for example, 6.0N/s to a load of, for example, 4.9N with the sensing element of the scaffold in tension while load, stroke, and strain measurements are recorded. Other loading rates and load values can be used, as appropriate. Then the potting fixture can be rotated to, for example, 180° and the test can be repeated at least six times with the sensing element of the strain gauge in compression. The strain values, for example, at 4.9N can be collected and averaged, and the percent strain transfer can be calculated by using the formula:

% strain transfer=[(strain_{experimental scaffold}−strain_{control scaffold})÷strain_{control scaffold}]*100

In some instances, the percent strain transfer, under tension or compression, of the scaffolds described can range from between about 20 to about 140%, as well as sub-ranges and individual percentage values contained within.

### IV. Methods of Making

The composite filaments can be prepared from a blend containing a bioresorbable polymer and an inorganic component using techniques known in the art. For example, the composite filaments can be prepared by infusing particles of the inorganic component into the bioresorbable polymer through a melted slurry, followed by an extrusion process on a filament extruder.

The customizable scaffolds can be prepared from the composite filaments using a commercially available 3D printer. After printing, the scaffolds can be sterilized using common techniques known in the art, such as ethylene oxide sterilization.

The shape and size of the scaffolds can be determined based on the configuration and pattern of the bone defect, which can be visualized using imaging techniques such as μCT. The scaffolds are generally designed based on the inverse structure of the bone with the defect. The subject having the bone defect can be scanned at the region of the bone defect using an imaging technique with a high resolution (e.g., 12 micron). The imaging data is then inverted so that images are created to show bone where space used to be and space where bone used to be. The images are then used to guide digital creation of the exterior of the scaffolds under a geometric pattern (e.g., a cylinder). This step can be performed using 3D design software, such as Solidworks. Then, 3D printing of the scaffolds can be performed based on the digital framework obtained from the prior step.

### V. Methods of Using

Methods of treating a bone defect in a patient generally include implanting a scaffold in the patient at the site of the defect. Optionally, prior to implantation, the scaffold is seeded with cells, such as stem cells, derived from the patient.

Optionally, prior to or at the time of implantation, an additional calcium mineral containing material is infused into the scaffold. The material can be in the form of fine particles. The calcium mineral containing material can be the same as or different from the inorganic component that is used in forming the composite filaments.

Suitable calcium mineral containing materials include but are not limited to ceramic compounds, such as biodegradable ceramic compounds have shown favourable properties in the matrix of the composite filaments. The ceramic compound preferably comprises a calcium mineral, like hydroxyapatite, calcium phosphate or calcium sulfate. Suitable materials include biodegradable porous mixtures of hydroxyapatite and tricalcium phosphate, like TRICOS® from Biomatlante (France) or CAMCERAM® from Cam Implants (Leiden, Netherlands). One option is a degradable mixture of hydroxyapatite and tricalcium phosphate (e.g., TRICOS®, which contains a mixture of 60% hydroxyapatite and 40% tricalcium phosphate, or CAMCERAM®).

Optionally the calcium containing material contains nonporous hydroxyapatite or tricalcium phosphate particles, pure hydroxyapatite particles (porous or nonporous), tricalcium phosphate particles (porous or nonporous), calcium sulfate powder, fine bone chips (either autograft or allograft) or xenograft bone chips.

The methods can be used to treat musculoskeletal diseases or traumas that require reconstruction of a calcified tissue (i.e., bone). The site in need of treatment can be located in a long bone, short bone, flat bone, sesamoid bone, irregular bone, trabecular bone, facial bone, or other bone in the body. The scaffold can be used for the repair and healing of bone fractures, in particular for bone fractures with a risk of becoming delayed unions or non-unions. A range of bone defects can benefit from the customizable scaffolds, which can be designed for a specific patient, based on factors including, age, activity level, profession, injury size, injury site, etc. Optionally the bone defect is a fracture, i.e., discontinuity or break across the entire bone structure creating two or more distinct bone segments, such as a fracture of the wrist (distal radius fractures), long bone fracture, like tibia, or hip fracture.

The bone defect can be the result of resection of a bone cysts or bone tumor.

In some cases, the bone defect is in a weight-bearing bone, especially a weight-bearing long bone such as tibia. In some cases, the bone defect is a critical sized bone defect.

The disclosed composite filaments, 3-D printed scaffolds, methods of making, and methods of treating disclosed above can be further understood through the following numbered paragraphs.

1. A composite filament for 3D printing, wherein the composite filament comprises a matrix formed of a blend comprising a bioresorbable polymer and an inorganic component.
2. The composite filament of paragraph 1, wherein the composite filament does not contain collagen.
3. The composite filament of paragraphs 1 or 2, wherein the composite filament consists essentially of the bioresorbable polymer and the inorganic component.
4. The composite filament of any one of paragraphs 1-3, wherein the blend comprises greater than 5% of the inorganic component by weight of the total mass of the matrix.
5. The composite filament of any one of paragraphs 1-4, wherein the bioresorbable polymer is elastic in its initial mechanical response.
6. The composite filament of any one of paragraphs 1-5, wherein the bioresorbable polymer is selected from the group consisting of polyalkenes, polyesters, polyurethanes, polyureas, polyanhydrides, polyamides, nylon 2 and higher nylons (such as nylon 6, nylon 12, and nylon-6,6), and blends and copolymers thereof.
7. The composite filament of paragraph 6, wherein the bioresorbable polymer comprises poly(lactic acid).
8. The composite filament of paragraph 6, wherein the bioresorbable polymer comprises thermoplastic polyurethane.
9. The composite filament of any one of paragraphs 1-8, wherein the bioresorbable polymer comprises poly(lactic acid) and thermoplastic polyurethane.
10. The composite filament of any one of paragraphs 1-9, wherein the inorganic component is a ceramic.
11. The composite filament of paragraph 10, wherein the inorganic component is a calcium phosphate ceramic.
12. The composite filament of any one of paragraphs 1-11, wherein the inorganic component comprises a tricalcium phosphate, a hydroxyapatite, or a combination thereof.
13. The composite filament of paragraph 12, wherein the inorganic component comprises beta tricalcium phosphate.
14. The composite filament of any one of paragraphs 1-13, wherein the bioresorbable polymer comprises poly(lactic acid), and the inorganic component comprises beta tricalcium phosphate.
15. The composite filament of any one of paragraphs 1-13, wherein the bioresorbable polymer comprises thermoplastic polyurethane, and the inorganic component comprises beta tricalcium phosphate.
16. The composite filament of any one of paragraphs 1-15, wherein the composite filament is elastic in its initial mechanical response.
17. A 3D-printed scaffold formed from the composite filament of any one of paragraphs 1-16.
18. The 3D-printed scaffold of paragraph 16, having a compressive modulus between about 40 and about 200 MPa.
19. The 3D-printed scaffold of paragraphs 17 or 18, having a shape and size configured for repairing a bone defect.
20. The 3D-printed scaffold of any one of paragraphs 17-19, wherein the 3D-printed scaffold is porous.
21. The 3D-printed scaffold of any one of paragraphs 17-20, having a maximum compression or tensile load of at least 1,500 N, at least 2,000 N, or at least 2,500 N.
22. A method of making a scaffold, comprising:

(a) 3D-printing the scaffold using the composite filament of any one of paragraphs 1-16 as a printing material.

23. The method of paragraph 22, further comprising after step (a):

(b) sterilizing the scaffold.

24. A method of treating a bone defect in a patient, comprising:

(a) implanting the 3D-printed scaffold of any one of paragraphs 17-21 in the patient.

25. The method of paragraph 24, further comprising prior to step (a), seeding the 3D-printed scaffold with cells derived from the patient.
26. The method of paragraph 25, wherein the cells are stem cells.
27. The method of any one of paragraphs 24-26, wherein the bone defect is a critical sized bone defect.
28. The method of any one of paragraphs 24-27, wherein the bone defect is in a weight-bearing bone.

### Examples

### Example 1. Preparation and Mechanical and Spectroscopic Assessment of PLA-Based Composite Filaments

Major injuries resulting in bone loss can occur from trauma or resection of bone during cancer treatment. No current surgical treatment reliably heals these defects, and each treatment has drawbacks that can include long periods of non-weight bearing and multiple surgeries. Biomimetic sensate polybutylene terephthalate (PBT) scaffolds in combination with TCP coatings were previously tested to heal large segmental bone defects (Szivek et al., J Biomed Mater Res B Appl Biomater, 2019, 107(2):242-252). While PBT was implanted as a component of the scaffolds, as a pure polymer it does not resorb.

This example produces biomimetic sensate scaffolds using a resorbable and implantable polymer, PLA, or PLA/TCP blends. Incorporation of tricalcium phosphate (TCP) particles within the PLA polymer will allow better control over the mechanical and biological properties.

Methods

PLA was mixed with TCP to produce filaments for 3D printing. PLA and TCP were mixed in one of three ratios (100:0, 85:15, 50:50) by weight and were converted to a filament using a Filabot EX2.

Raman spectroscopy was performed on the PLA/TCP composite filaments and the scaffolds made from these filaments. Scanning electron microscopy (SEM) was performed on the PLA/TCP composite filaments to obtain both a lateral view and a cross-section view. To obtain the cross-section view, the filaments were broken after immersion in liquid nitrogen to obtain a clean break of the filaments.

The PLA and PLA/TCP filaments were used to print a 3D biomimetic scaffold based on trabecular patterns collected from μCT scans of sheep femoral heads. All scaffolds produced had a diameter of 2.5 cm and a height of 1.8 cm. PLA and PLA/TCP based scaffolds were produced using a Flashforge Creator Pro printer. Polybutylene terephthalate (PBT) scaffolds, with and without a TCP coating, were produced using a Stratasys fused deposition modeler, as controls. The PBT-based scaffolds were previously used to regenerate large segments of bone.

Five types of scaffold materials were prepared and tested: PBT, PBT/TCP, PLA, 85:15 PLA/TCP, and 50:50 PLA/TCP.

Thermogravimetric analysis (TGA) was used to analyze the materials used to produce each type of scaffold. A small sample (ca. 5 mg) from each type of scaffold was used for the analysis. TGA was performed using a TA Instruments Thermogravimetric Analyzer by heating the sample at 10° C. per minute up to 700° C.

Prior to mechanical testing, strain gauges were attached to the scaffolds. Mechanical testing was performed in compression by loading at 2 N/sec to a maximum load of 2000 N or failure. Additional non-destructive mechanical testing was performed to determine whether mechanical properties of the scaffolds changed with time following exposure to a simulated in vivo environment. After initial testing, the scaffolds were stored in Dulbecco's Modified Eagle's Medium with 10% Fetal Bovine Serum at 37° C. and 5% CO₂ for 4 weeks. During this time, scaffold weight was recorded weekly and mechanical testing was repeated at the end of the 4 weeks. Scaffold stiffness was compared in SPSS using multivariate analysis and a Tukey post-hoc test to analyze the effect of scaffold type and time on mechanical properties.

Results

PLA-based filaments with a diameter in the range between about 1.65 and about 1.8 mm were produced. Spectroscopic characterizations of the filaments showed that large amounts of the ceramic TCP were part of the PLA/TCP filaments. SEM imaging showed an even distribution of TCP in the PLA/TCP filaments Raman spectroscopic study on the PLA/TCP filaments showed a change in chemical bond strength and type compared to a mixture of the starting materials of the filaments.

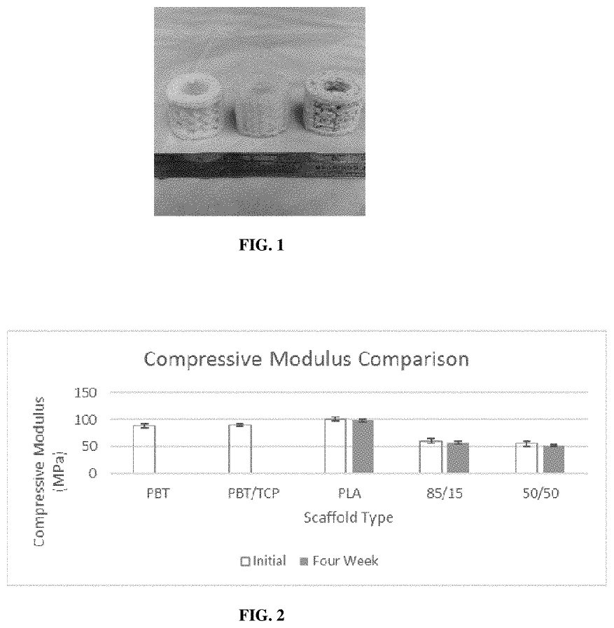

Scaffolds were produced using PLA or PLA/TCP filaments. The scaffolds had the same structure as the previously produced PBT and PBT/TCP scaffolds (FIG. 1).

TGA showed that the 50:50 PLA/TCP filament resulted in scaffold containing 28-32 volume % TCP. Although the 50:50 PLA/TCP filament showed a lower ceramic content following the scaffold production process, TGA of the 85:15 PLA/TCP filament demonstrated that the final scaffold contained 15-18 volume % TCP.

No scaffolds produced from PBT or with blended PLA/TCP failed under the 2000 N load limit during compressive mechanical testing. The pure PLA scaffolds failed at 1232±94 N. All scaffolds demonstrated linear elastic deformation in compressive testing. Initial scaffold stiffness was 88.61±4.14 MPa for PBT scaffolds, 89.52±3.23 MPa for PBT/TCP scaffolds, 100.53±3.51 MPa for PLA scaffolds, 59.99±4.61 MPa for 85:15 PLA/TCP scaffolds, and 55.19±5.24 MPa for 50:50 PLA/TCP scaffolds (FIG. 2). Pure PLA scaffolds had the highest stiffness while no changes in stiffness were noted between the PBT and PBT/TCP scaffolds. Addition of TCP to PLA significantly decreased stiffness, but no significant differences were noted between the PLA/TCP blends. During exposure to a simulated in vivo environment for 4 weeks, no changes in scaffold mass or stiffness were noted in any of the scaffolds printed for this batch (FIG. 2).

TGA analysis of the PLA/TCP scaffolds demonstrated that the scaffold production process may decrease the TCP content in scaffolds produced with a 50:50 blend of TCP and PLA. Mechanical testing demonstrated that all scaffolds had sufficient strength for use in a previously developed in vivo sheep model (Szivek et al., J Biomed Mater Res B Appl Biomater, 2019, 107(2):242-252), allowing for full immediate weight bearing. Application of a TCP coating to PBT did not alter mechanical properties. Introduction of TCP into the PLA filament allowed for manipulating the mechanical properties of the scaffold as this decreased the compressive stiffness and resulted in improved failure load. While PLA-based scaffolds were expected to degrade in vitro in this experiment, no appreciable changes in mass or mechanical properties were noted following one month of incubation at simulated in vivo conditions.

### Example 2. In Vitro Cell Culture Testing of PLA-Based and TPU-Based Composite Filaments

Methods

Biomimetic scaffolds were produced using a Stratasys 1650 FDM printer or a Flashforge Creator Pro printer, based on the trabecular patterns of sheep femoral heads determined by μCT scans. The biomimetic scaffolds measuring 10 mm diameter×5 mm height were made. PBT scaffolds, with and without a TCP coating, were used as controls. PLA scaffolds were printed from pure PLA, or PLA blended with 25% TCP (75/25 PLA/TCP) or 50% TCP (50/50 PLA/TCP) by weight. Thermoplastic urethane (TPU) scaffolds were printed from pure TPU, or TPU blended with 25% TCP (75/25 TPU/TCP) or 50% TCP (50/50 TPU/TCP) by weight. A total of 48 scaffolds were made, 6 of each material (PBT, PBT/TCP, PLA, 75/25 PLA/TCP, 50/50 PLA/TCP, TPU, 75/25 TPU/TCP, 50/50 TPU/TCP). After fabrication, scaffolds were sterilized using ethylene oxide.

Scaffolds were seeded with 5×10⁴ human adipose derived stem cells (ADCs) per scaffold and maintained in STEMPRO™ osteogenic media, which was exchanged three times per week. At 7 and 14 days, cell viability was measured using an XTT assay according to instructions from the CYQUANT™ XTT assay kit, and osteoblastic differentiation was assessed with an alkaline phosphatase (ALP) assay, according to instructions from the ABCAM® ALP assay kit. ALP activity was normalized to the cell count. ALP activity indicates calcified tissue formation. A high ALP levels indicates ongoing bone formation.

Data was compared using ANOVA and a Tukey post-hoc test in SPSS.

Results

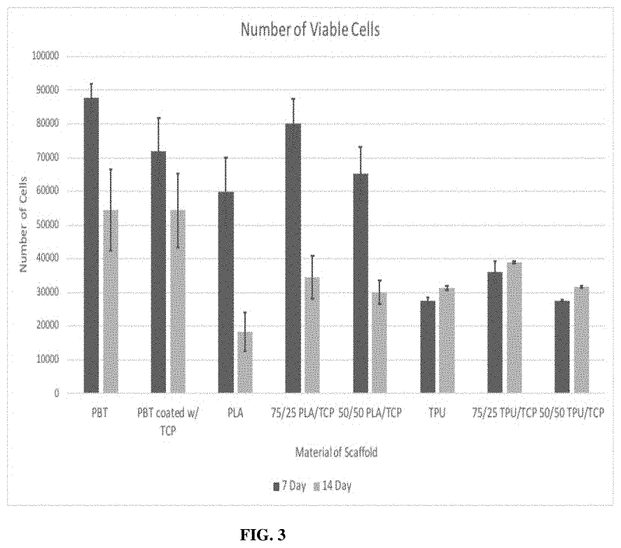

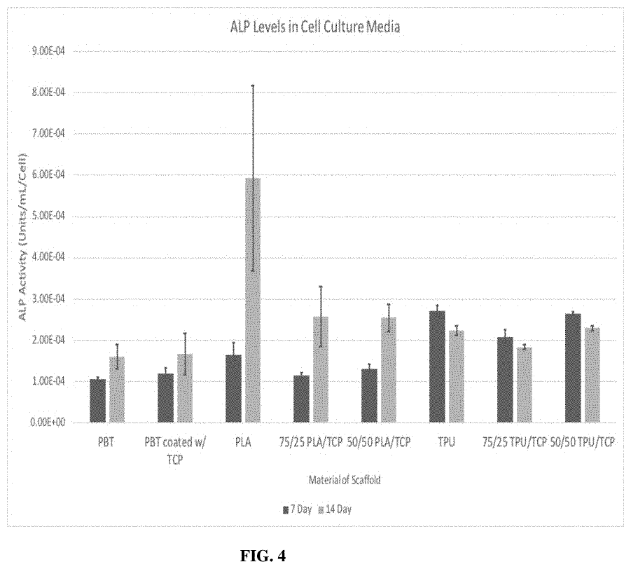

Scaffolds produced with each material supported cell adhesion and viability at all time points. Scaffolds made from pure PBT or PLA demonstrated fewer cells at 2 weeks, while TPU-based scaffolds demonstrated the same cell viability over time (FIG. 3). At 2 weeks, the ALP activity was higher in all PLA-based scaffolds compared to PBT-based scaffolds (FIG. 4). Pure PLA scaffolds demonstrated significantly higher ALP activity compared to all other scaffolds (p<0.005) at 2 weeks.

Thus the scaffolds supported ADC attachment and osteoblastic differentiation. Pure PLA scaffolds showed lower cell viability after 2 weeks but demonstrated significantly increased ALP levels compared to other scaffolds. At 2 weeks, the PLA/TCP, TPU, and TPU/TCP scaffolds showed less cell viability, but increased ALP levels compared to PBT and PBT/TCP scaffolds.

Thus, the PLA/TCP, TPU, and TPU/TCP scaffolds had cells that showed a trend toward increased osteoblastic phenotype compared to cells on the PBT-based scaffolds after 2 weeks.

### Example 3. In Vitro Cell Culture Testing of PLA-Based and TPU-Based Composite Filaments

Methods

75:25 PLA:TCP and 50:50 PLA:TCP biomimetic scaffolds were produced as rectangular implants 10 mm long×5 mm wide×2 mm thick, according to methods of Example 2 above. The biomimetic scaffolds contained uniform porous channels (approximately 400 μm diameter). The biomimetic scaffolds had a uniaxial 120Ω, strain gauge attached to one surface. The strain gauge was waterproofed and the scaffolds were sterilized using ethylene oxide.

Scaffold design and mechanical testing parameters were based on a previous study with modifications that included placement of one scaffold type per rat, use of a longer scaffold, no protein growth factors (TGF-β1), and a three-month endpoint (Szivek J A, et al., TGF-β1 enhanced TCP coated sensate scaffolds can detect bone bonding. Journal of Biomedical Materials Research, 73B(1); 43-53, 2005).

Latex coated PBT scaffolds were used as one of two control scaffolds. The PBT scaffolds are currently used for regeneration of large bone defects in a sheep model, and have been previously tested in rats. Pure PLA scaffolds served as a second control. Composite ceramic polymer biomimetic scaffolds made of 75:25 PLA:TCP and 50:50 PLA:TCP, as described herein, were implanted as the experimental scaffolds in rats.

Each rat in this study received one scaffold (PBT, PLA, or composite ceramic polymer biomimetic scaffold) that was circumferentially held in place on the anterior surface of a femur using vicryl sutures. After three months both femora were explanted. A scaffold of the same type as the implanted scaffold was glued to the control bone in the same location and orientation using epoxy. The bones were potted in a test fixture using Cerrobend so the strain gauge was aligned with the loading axis of the test fixture. The bones were then loaded six times at a rate of 6.0N/s to a load of 4.9N with the sensing element of the scaffold in tension while load, stroke, and strain measurements were recorded. The potting fixture was rotated 180° and the test was repeated six times with the sensing element of the strain gauge in compression. The strain values at 4.9N were collected and average, and the percent differences were calculated by using the formula:

% strain transfer=[(strain_{experimental scaffold}−strain_{control scaffold})÷Strain_{control scaffold}]*100

Further, the bone volume (BV) over total volume (TV) ratio (BV/TV) of the implanted PBT, PLA, and composite ceramic polymer biomimetic scaffolds was determined using computerized tomography (CT) measurements. For the implanted scaffolds, the BV/TV ratio is listed in the table below:

TABLE 1

| Material | Bone Volume (mm3) | BV/TV (%) |
|---|---|---|
| PBT | 70.21 +/− 8.03 | 65.65 +/− 4.68 |
| PLA | 71.09 +/− 7.08 | 65.49 +/− 3.76 |
| 75-25 | 74.93 +/− 7.45 | 69.15 +/− 3.72 |
| 50-50 | 74.93 +/− 6.66 | 63.76 +/− 3.76 |

As shown in the table above, the BV/TV values of the scaffolds indicated that all scaffolds appear to encourage about the same amount of new bone formation. In summary, it was found that all the scaffold materials tested including the PLA and blended plastics (75:25 PLA:TCP and 50:50 PLA:TCP), which are biodegradable, appear to induce at least about the same amount of bone growth as the PBT scaffold, which is not biodegradable, and has been shown to be a useful scaffold material for bone implant applications.

1. A composite filament for 3D printing, wherein the composite filament comprises a matrix formed of a blend comprising a bioresorbable polymer and an inorganic component.

2. (canceled)

3. The composite filament of claim 1, wherein the composite filament consists essentially of the bioresorbable polymer and the inorganic component.

4. The composite filament of claim 1, wherein the blend comprises greater than 5% of the inorganic component by weight of the total mass of the matrix.

5. (canceled)

6. The composite filament of claim 1, wherein the bioresorbable polymer is selected from the group consisting of polyalkenes, polyesters, polyurethanes, polyureas, polyanhydrides, polyamides, nylon 2, nylon 6, nylon 12, nylon-6,6, and blends and copolymers thereof.

7. The composite filament of claim 6, wherein the bioresorbable polymer comprises poly(lactic acid).

8-9. (canceled)

10. The composite filament of claim 1, wherein the inorganic component is a ceramic.

11. The composite filament of claim 10, wherein the inorganic component is a calcium phosphate ceramic.

12. The composite filament of claim 10, wherein the inorganic component comprises a tricalcium phosphate, a hydroxyapatite, or a combination thereof.

13. (canceled)

14. The composite filament of claim 1, wherein the bioresorbable polymer comprises poly(lactic acid), and the inorganic component comprises beta tricalcium phosphate.

15-16. (canceled)

17. A 3D-printed scaffold formed from the composite filament of claim 1.

18-30. (canceled)

31. The 3D-printed scaffold of claim 17, having a compressive modulus between about 40 and about 200 MPa.

32. (canceled)

33. The 3D-printed scaffold of claim 17, wherein the 3D-printed scaffold is porous.

34. The 3D-printed scaffold of claim 17, having a maximum compression or tensile load of at least 1,500 N, at least 2,000 N, or at least 2,500 N.

35. A method of making a scaffold, comprising:
(a) 3D-printing the scaffold using the composite filament of claim 1 as a printing material.

36. The method of claim 35, wherein the blend comprises greater than 5% of the inorganic component by weight of the total mass of the matrix.

37. (canceled)

38. The method of claim 36, wherein the bioresorbable polymer is selected from the group consisting of polyalkenes, polyesters, polyurethanes, polyureas, polyanhydrides, polyamides, nylon 2, nylon 6, nylon 12, nylon-6,6, and blends and copolymers thereof.

39. The method of claim 38, wherein the bioresorbable polymer comprises poly(lactic acid).

40-41. (canceled)

42. The method of claim 35, wherein the inorganic component comprises:
(1) a ceramic;
(2) a calcium phosphate ceramic; or
(3) a beta tricalcium phosphate.

43-45. (canceled)

46. The method of claim 35, wherein the bioresorbable polymer comprises poly(lactic acid), and the inorganic component comprises beta tricalcium phosphate.

47-49. (canceled)

50. A method of treating a bone defect in a patient, comprising:
(a) implanting the 3D-printed scaffold of claim 17 in the patient.

51-54. (canceled)
